(19) United States
(12) Patent Application Publication
(10) Pub. No.: US 2026/0325184 A1
(43) Pub. Date: Oct. 22, 2026

(54) MINIMALLY INVASIVE SYSTEM FOR DYNAMIC COMPRESSION OF BONE SEGMENTS

(71) Applicant: Forma Medical, Inc., Camp Hill, PA (US)

(72) Inventors: James A. Gault, Lincoln, RI (US); Andrew Charles Davison, Hummelstown, PA (US)

(73) Assignee: Forma Medical, Inc., Camp Hill, PA (US)

(21) Appl. No.: 19/648,710

(22) Filed: Apr. 15, 2026

Related U.S. Application Data
(60) Provisional application No. 63/789,652, filed on Apr. 16, 2025.

(51) Int. Cl. A61B 17/80; A61B 17/00; A61B 17/68; A61B 17/88

(52) U.S. Cl. CPC A61B 17/8019; A61B 17/00234; A61B 17/808; A61B 17/88; A61B 2017/00238; A61B 2017/681

(57) ABSTRACT

A system configured to guide one or more wires or fixation elements toward one or more bone segments of extremity bones is provided.

### CROSS-REFERENCE TO RELATED APPLICATION

The present application claims priority under 35 U.S.C. § 1.19(e) to and the benefit of U.S. Provisional Application No. 63/789,652, filed Apr. 16, 2025, the entire contents of which are incorporated herein by reference.

### TECHNICAL FIELD

The present disclosure relates to a minimally invasive system for dynamic compression of bone segments and related methods.

### BACKGROUND

Dynamic compression has been proven to be beneficial in certain anatomic regions where continuous compression is desired. For extremity bones this typically has been achieved through the use of staples, like Medline's Unite Reflex staples and Treace's SpeedPlate. Conventional solutions for dynamic compression that exist today are monolithic. This means that the incision must be at least as long as the device and therefore cannot be used in a minimally invasive approach.

### SUMMARY

An embodiment of the present disclosure includes a minimally invasive system for compression of bone segments or fragments in an extremity bone. The system includes a bone palate having a trailing end, a leading end spaced from the trailing end, and a length that extends from the trailing end to the leading end. The bone plate is curved along the length and is configured to flex. The system includes a first elongated element configured to be coupled to the trailing end of the bone plate, and a second elongated element is configured to be coupled to the leading end of the bone plate. The system includes a device configured to be coupled to the first elongated element and the second elongated element while the first and second elongated elements are coupled to the bone plate. The minimally invasive system has 1) a first configuration where the first elongated element is coupled to the trailing end of the bone plate and the second elongated element is coupled to the leading end of the bone plate so that the first elongated element and the second elongated element are splayed apart with respect to each other, and 2) a second configuration when the device is coupled to proximal ends of the first elongated element and the second elongated element so that the first elongated element and the second elongated element are no longer splayed apart with respect to each other and the bone plate is less curved or no longer curved along the length.

Another embodiment of the present disclosure is a method that includes positioning a bone plate through a first incision with a first elongated element coupled to the bone plate so that the bone plate spans bone segments. The method includes forming a second incision in a location spaced from the first incision. The method includes coupling a second elongated element to the bone plate through the second incision, and coupling a device to the first elongated element and the second elongated element to flex the bone plate from a first configuration into a second configuration. The method includes inserting bone anchors through the first and second incisions to couple the bone plate to bone segments. The method also includes removing the device from the first elongated element and the second elongated element to transition from the second configuration toward the first configuration to compress the bone segments together.

Another embodiment of the present disclosure is a method that includes forming a first incision in tissue near a joint or fracture between bone segments. The method includes coupling a first elongated element to a trailing end of a bone plate, the bone plate having a first curved configuration. The method includes inserting a leading end of the bone plate through the first incision until the leading end of the bone plate is adjacent a first bone segment and a trailing end is adjacent to a second bone segment. The method includes forming a second incision in tissue near where the leading end is located. The method includes a coupling, through the second incision, a second elongated element to the leading end of the bone plate adjacent to the second bone segment. The method further includes coupling a jig to proximal ends of the first elongated element and the second elongated element to transition the bone plate from the first configuration into a second configuration that differs from the first configuration. The method includes inserting a first anchor, through the first incision, into a first outer hole in the trailing end of the bone plate and into the first bone segment. The method includes inserting a second anchor, through the second incision, into a second outer hole in the leading end of the bone plate and into the second bone segment. The method includes removing the jig from the proximal ends of the first elongated element and the second elongated element to transition from the second configuration toward the first curved configuration, thereby compressing the first bone segment and the second bone segment together.

### BRIEF DESCRIPTION OF THE DRAWINGS

The foregoing summary, as well as the following detailed description of exemplary embodiments of the present application, are better understood when read in conjunction with the appended drawings. For the purposes of illustrating the present application, shown in the drawings are exemplary embodiments of the disclosure. It should be understood, however, that the application is not limited to the precise arrangements and instrumentalities shown. In the drawings:

FIG. 1A is a top perspective view of a bone plate according to an embodiment of the present disclosure;

FIG. 1B is a side view of the bone plate shown in FIG. 1A;

FIG. 1C is a top view of the bone plate shown in FIG. 1A;

FIG. 1D is an end view of the bone plate shown in FIG. 1A;

FIG. 1E is side view of a bone plate shown in FIG. 1A, illustrating the bone plate in a normal bent configuration and a generally straight configuration after the bone plate is flexed;

FIG. 2A is a perspective view of a device configured to facilitate compression according to an embodiment of the present disclosure;

FIG. 2B is a bottom end view of the device shown in FIG. 2A;

FIG. 3 is a perspective view of an elongated element;

FIGS. 4A and 4B illustrate a side view of bone anchors according to an embodiment of the present disclosure.

FIG. 5 illustrate forming a first incision in tissue near bone segments;

FIG. 6 illustrates elongated elements coupled to bone plate according to an embodiment of the present disclosure and formation of a second incision into the tissue;

FIG. 7 illustrates insertion of bone plate and elongated element through the first incision;

FIG. 8 illustrates coupling an inside elongated element to the bone plate through the second incision;

FIG. 9 illustrates a jig coupled to proximal ends of the elongated elements to flex the bone plate into a straight configuration;

FIG. 10 illustrates use of stabilizing wire to stabilize position of the bone plate relative to the bone segments;

FIG. 11 illustrates placement of outside elongated elements into the outside holes of the bone plate through first and second separate incisions;

FIGS. 12 and 13 illustrate removal of outside elongated element and insertion of outside bone anchors into the outside holes of the bone plate to fix the bone segments in a compressed state, with FIG. 13 shows the tissue removed for illustrative purposes;

FIGS. 14 and 15 illustrates a removal of the jig from the first and second interior elongated elements with FIG. 15 show the tissue removed for illustrative purposes;

FIG. 16 illustrates the bone plate fixed to compressed bone segments; and

FIGS. 17-19 illustrates a method for compressing and fix bone segments using a jig according to an embodiment of the present disclosure.

FIG. 20 illustrates a fixation instrument according to an embodiment of the present disclosure.

FIG. 21 illustrates a device for aligning elongated elements into the bone plate according to an embodiment of the present disclosure.

FIG. 22 illustrates an exploded view of the device of FIG. 21 according to an embodiment of the present disclosure.

FIG. 23 illustrates a cross-section of the device of FIG. 21 according to an embodiment of the present disclosure.

### DETAILED DESCRIPTION OF ILLUSTRATIVE EMBODIMENTS

Systems described in this specification are configured to aid in the fixation of two or more bones or bone segments on either side of a joint or fracture of bone segments, typically in extremity bones, such as the foot, and to compress adjacent bone segments. The systems described below can be used in any extremity bone, such as the hand and foot or in other areas of the body as need. For foot, the system can be used in various skeletal anatomy of a foot includes, including the tarsals, metatarsals, and phalanges. The system could be used in in of the three typically defined regions of foot bone structure: the hindfoot, midfoot, and forefoot. The tarsal bones are seven bones in the hindfoot and midfoot and include the calcaneus, talus, cuboid, navicular, and three cuneiforms. The metatarsal bones are five bones in the forefoot that connect the tarsals to the phalanges. The phalanges in the forefoot that form the toes. The systems as described herein are configured for fixation of toe and/or for hammertoe correction, for example. In other examples, the systems and methods described herein are configured for interphalangeal joint fixation. For example, the systems may be used for, or to aid in fixation of metatarsals, proximal phalanges, middle phalanges, or distal phalanges. While the embodiments described are configured for interphalangeal joint fixation, it is possible that the described embodiments could be configured for fixation of phalanges, metatarsals, cuneiform, or cuboid bones in the foot. In other embodiments, systems may be used for fixation of bone segments of phalanges, metatarsals, or other bones in the hand, or elsewhere as needed based on context and anatomical applicability.

The present disclosure uses terms like distal and proximal to indicate reference to the devices and system components being described. The term “distal” shall mean away from the center of a body (or device). The term “proximal” shall mean closer towards the center of a body (or device) and/or away from the “distal” end. Thus, proximal typically means closer to the user or surgeon and the distal refers to the further away from the user or surgeon, relative to the device, instrument or anchor, etc.

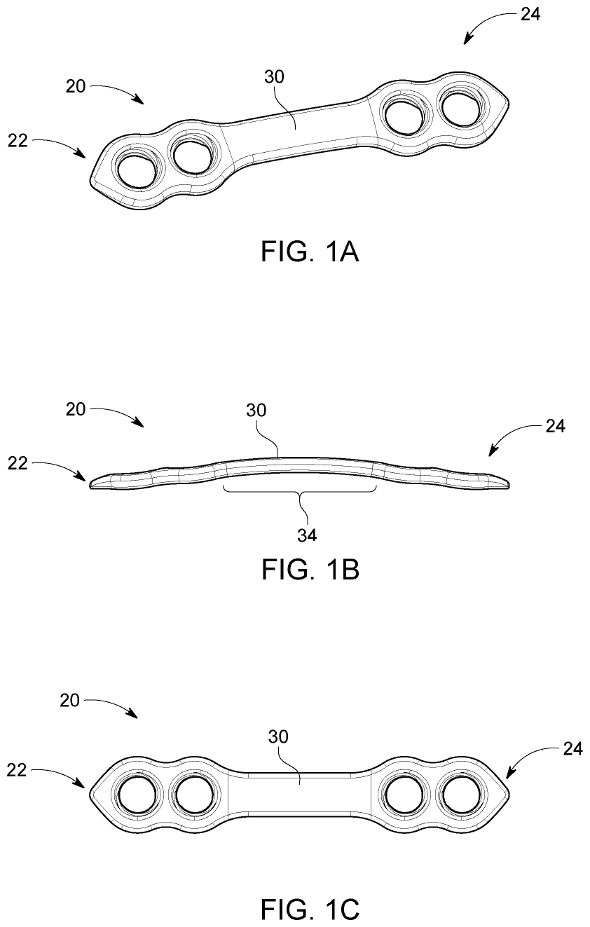

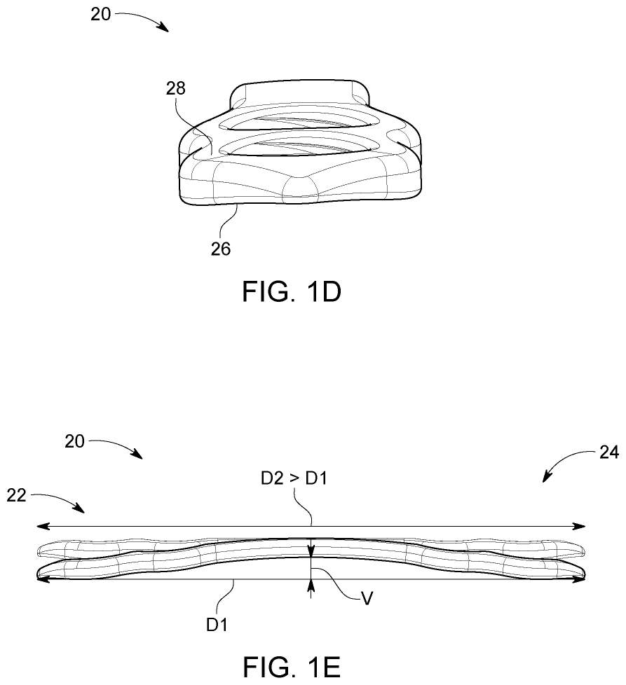

As shown in FIG. 1A-4B, a minimally invasive system 10 for extremity bones includes a curved bone plate 20, sets of elongated elements 40, 50, 60, 70, a device 80 configured to couple to the elongated elements and used to compress the bone segments, and bone anchors 300, 350. The system 10 as described herein may be used to, in a minimally invasive fashion, insert the bone plate over bone segments, compress the bone segments, and fix the bone plate 20 to bone segments to aid in bone fusion or stabilization. More specifically, the bone plate 20 may be inserted, with one or more elongated elements coupled thereto, through a small incision. Once the bone plate is in place, another elongated element is coupled to the bone plate through a separate small incision. Because the bone plate is curved, the elongated elements when coupled to the bone plate have a splayed configuration. A device is coupled to the elongated elements, which flexes the bone plate into a straighter configuration, so the elongated elements are no longer splayed apart and the bone segments are compressed. Bone anchors are then coupled the bone plate and bone segments to fixed the place. When the device is removed, the bone anchors fixed to bone compress the bone segments and maintain the bone plate is second straighter configuration. Details of this system and method of use are discussed in further below.

Referring to FIGS. 1A-1E, the bone plate 20 is configured to span a joint or fracture between adjacent bone segments or bone segments. The bone plate 20 has a trailing end 22, a leading end 24 spaced from the trailing end 22, an inferior surface 26 configured to face the bone, and a superior surface 28 opposite the inferior surface 26. A central bridge 30, which does not typically include any holes or the like, extends between the leading end 24 and the trailing end 22. The bone plate 20 has a length that extends from the trailing end 22 to the leading end. Here, the bone plate 20 is curved along the length. The bone plate, however, is flexible and responsive to external forces to bend from the curved, first configuration, into a straighter state.

The bone plate 20 is configured to be fixed to bone using bone anchors 300 or 350. Accordingly, the trailing end 22 of the bone plate includes a first hole and a second hole sized and configured to receive bone anchors. The leading end 24 of the bone plate 20 also includes a first hole and a second hole sized configured to receive bone anchors. The holes may be referred to as inner and outer holes depending on their location relative to the central bridge. The outer hole is generally opposite the central bridge 30 or portion of bone plate 20 and the interior hole is located between the outer hole and the central bridge. Each end of the bone plate 20 would therefore have an interior hole and an outer hole so that four bone anchors can be used for fixing the bone plate to the bone segments. The inner surface of bone plate 20, that defines the holes, are threaded as means to couple the elongated elements to the bone plate 20 and to receive any threaded heads of bone anchors as needed.

As shown in FIGS. 1B and 1E, bone plate 20 is curved between the trailing end 22 and leadings end, in its natural state, as shown in FIG. 1A. However, an external force can be used to flex the bone plate 20 from a curved, first configuration as shown in FIG. 1A, into a second, generally straight configuration, as shown in FIG. 1E. The transition of the bone plate 20 when fixed to the bone segments aides compressing the bone segments together, as described further below.

Continuing with FIGS. 1A-1E, the bone plate 20 as shown can include curvature along an entirety or just a portion of the length of the bone plate 20. For example, the entire length of the bone plate may be curved. In another example, the central bridge 30 is curved. In its natural state (FIG. 1A) the outer most surfaces of the leading end 24 and trailing end 22 form a distance D1 (e.g. a chord distance). The distance D1 is measured from along a straight-line from outer surfaces of the leading end 24 to the trailing end 22, respectively. A versine Vis a line segment drawn perpendicular to the distance D1 (or chord), located at the midpoint of distance D1 and the arc of the bone plate 20. The versine V of the bone plate 20 in this case will decrease as the bone plate 20 flexes, in use, as further described below. Likewise, the chord distance D1 increases to D2 when flexed. In the first configuration, the versine of the bone plate 20 can range between 0.5 mm and 5 mm, depending on the length of the bone plate 20. In one example, the bone plate 20 has a curved portion 34 that extends from the first inner hole to the second inner hole across the central bridge 30. The curved portion of the bone plate 20 may have a radius of curvature between about 20 to 900 mm. When the bone plate is flexed from its first configuration, as shown in FIG. 1D, the distance between the outermost surfaces of the leading end 24 and trailing end 22 increases as result of the curvature decreasing with increasing flexion.

In some embodiments, the inferior surface 26 of the bone plate 20 is curved to conform to the anatomy of the foot bone against which it is placed. The superior surface 28 may track the same or substantially similar curvature of the inferior surface 26.

The bone plate 20 includes a central portion (or central bridge 30) configured to span a joint, a proximal fixation portion extending from and angularly offset with respect to the central bridge 30, and a distal fixation portion extending from and angularly offset with respect to the central bridge 30. The central bridge 30 extends along a bridge axis.

The bone plate 20 may have a proximal end at the proximal fixation portion that is tapered, and a distal end at the distal fixation portion that is tapered. The tapered proximal and distal ends are configured to guide the bone plate 20 between skin and bone proximate and across joint during insertion to the fixation site. The proximal end may be referred to the leading end 24 of the bone plate 20 and the distal end may be referred to as the trailing end 22 of the bone plate 20, depending on direction of insertion. Likewise, the proximal end of the bone plate may be referred to as the trailing end of the bone plate and the distal end of the bone plate may be referred to as the leading end of the bone plate. Thus, the two opposed ends may be referred to a proximal, distal, trailing, or leading based on context.

A proximal fixation portion has one or more through-holes that extend from the superior surface to the inferior surface. The proximal fixation portion has first and second through-holes, which include partially threaded inner walls. Thus, the first and second through-holes can receive either locking bone anchors or compression anchors. The through-holes of the proximal fixation portion extend along a through-hole axis and include an upper portion that is tapered inward toward the through-hole axis. The one or more through-holes are generally aligned along a first axis.

A distal fixation portion has one or more through-holes that extend from the superior surface 28 to the inferior surface 26. The distal fixation portion has first and second through-holes, which include partially threaded inner walls as shown. Thus, the first and second through-holes can also receive either locking bone anchors or compression anchors. The through-holes of the distal fixation portion extend along a through-hole axis and include an upper portion that is tapered inward toward the through-hole axis. While the proximal fixation portion and distal fixation portion each include two through-holes, more or less through-holes may be used in each portion of the bone plate 20. The one or more distal through-holes are aligned along a second axis.

The bone plate 20 may include sets of bores (not shown) for receiving wires, such as k-wires or fixation pines. These bores may be placed between two adjacent through-holes and/or between a through-hole and the proximal and distal ends of the plate, respectively.

The central bridge 30 offsets the through-holes in proximal and distal fixation portions of the bone plate 20 such that the first axis and the second axis do not intersect. For example, the first axis and the second axis may be parallel. Furthermore, the central bridge 30 extends along the bridge axis and forms oblique angles with the first axis and the second axis. In some embodiments, these configurations result in a bone plate that can span a fixation site and allow fixation with bone anchors so that the anchors fall on either side of the extensor hallucis longus (EHL) tendon when the bone plate 20 is implanted.

Elongated elements as described in the present application may be used to guide instruments to drill holes into the bone and to provide a mechanism to compress the bone segments via interaction with the bone plate 20. In the example shown the elongated elements may be drill guides. The drill guide can be an elongated structure with a cannulation through which an instrument can pass. In this case, the system includes a set of interior drill elongated elements (or drill guides) 40, 50 and a set of outer elongated elements (or drill guides) 60, 70. A drill guide will described below but this is not a limiting description; rather it is used only to aid in the description of the invention. In this case, the interior drill guides include a first interior drill guide 40 and second interior drill guide 50 and the outer drill guides include a first outer drill guide 60 and a second outer drill guide 70. The interior drill guides 40, 50 are configured to engage the interior holes of the bone plate 20. The outer drill guides 60, 70 are configured to engage the outer holes of the bone plate, at opposite end of the bone plate 20. The outer drill guides 60, 70 are constructed in a similar way to drill guides 40, 50, although the outer drill guides 60, 70 are generally shorter in length compared to the interior drill guides. Accordingly, drill guide 40 is described below for clarity and descriptive purpose.

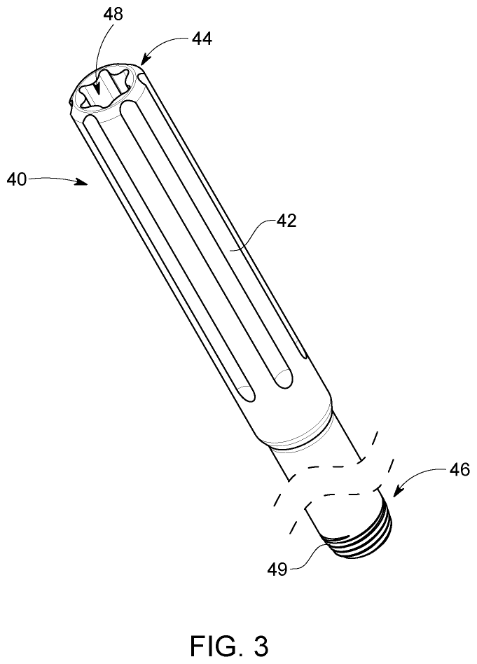

As shown in FIG. 3, drill guide 40 configured to engage the holes, 140 of the bone plate 20. The drill guide 40 may be an elongated body 42 that has a proximal end 44, a distal end 46 spaced apart from the proximal end 44, and a cannulation 48 that extends from the proximal end 44 to the distal end 46. The cannulation 48 is configured to receive a drilling instrument or other device to aid in bone preparation. The proximal end 44 may include an engagement feature (not labeled) to receive a driver. The distal end 46 includes a tip 49 configured to threadably engage the threads at the holes in the bone plate 20. The drill guides 40 (and drill guide 50, 60, and 70) are configured to threadably engage the bone plate 20. While threaded coupling between the drill guide and bone plate is shown and described, other means of coupling can be used such as press-fit connections, tabs, or the mechanical engagement feature for connection. In addition, while the drill guides are shown coupled to the holes because this allows bone preparation and holes to formed where the bone anchors will be placed, the drill guide could be coupled to other features or parts of the bone plate. Likewise, other devices or elongated elements may be used to couple to the bone plate, at the holes or in other locations to aid in inserting the bone plate through the incision and to facilitate flexing the bone plate via the device 80.

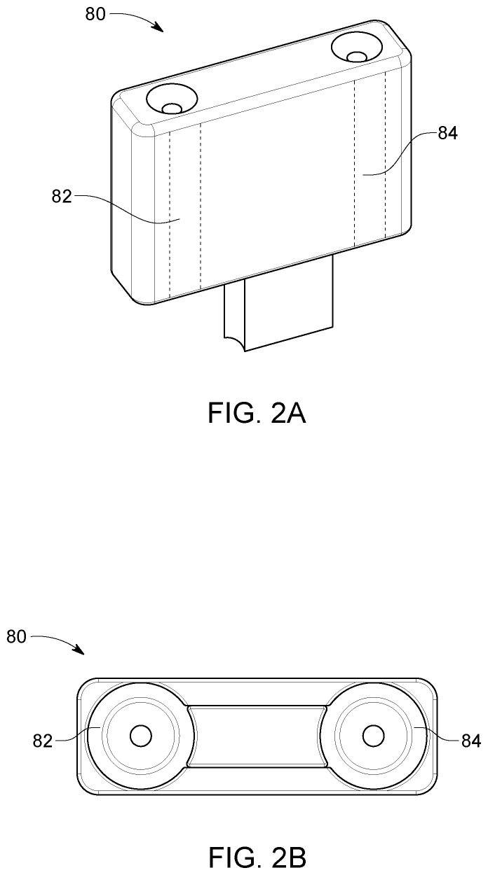

Referring to FIGS. 2A and 2B, the system also includes a device 80 configured to aid in compression bone segments via drill guides coupled to the bone plate 20. The device 80 could be any structure or device that can engage the drill guides when coupled to the bone plate 20. Accordingly, the device 80 includes one or more engagement elements 82, 84 for coupling to the first interior drill guide 40 and second interior drill guide 50. As such, the device 80 could be any structure, such as a block, plate, or band that can be used to engage the drill guides (when coupled the bone plate) when splayed apart and hold them in a non-splayed state as shown in the figures. Accordingly, the system has 1) a first configuration where the first interior drill guide 40 is coupled to the trailing end 22 of the bone plate 20 and the second interior drill guide 50 is coupled to the leading end 24 of the bone plate 20 so that the first interior drill guide 40 and the second interior drill guide 50 are splayed apart with respect to each other, and 2) a second configuration when the device 80 is coupled to proximal ends of the first and second interior drill guides 40,50 so that first interior drill guide 40 and the second interior drill guide 50 are no longer splayed apart with respect to each and the bone plate 20 is no longer curved.

As shown in FIGS. 2A and 2B, the device 80 is a jig or compression jig. The jig includes body having a forward surface and an opposite rearward surface. The forward surface faces the tissue T and bone plate when coupled to the elongated elements. The jig also includes a first engagement element configured to receive the first drill guide 40 and a second engagement element separate from the first second engagement element configured to receive the second elongated element 40. In this example, the first engagement element is a first channel, and the second engagement element is a second channel. The device 80 may also include features for helping a first bore at the first configured to receive a stabilization wire and a second bore configured to receive another stabilizing wire.

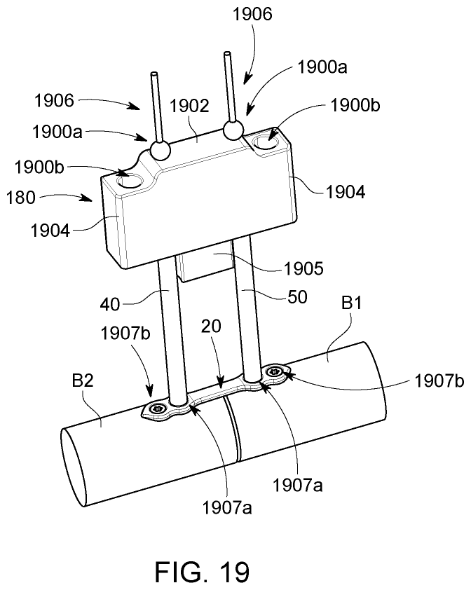

In alternative designs (FIGS. 17-19), the device 180 has a first engagement elements, a second engagement element, a third engagement element, and a fourth engagement element. Like described above, the third engagement element may be a third channel and the fourth engagement element is a fourth channel, with each channel sized and shaped to receive first and second outer drill guides 60,70.

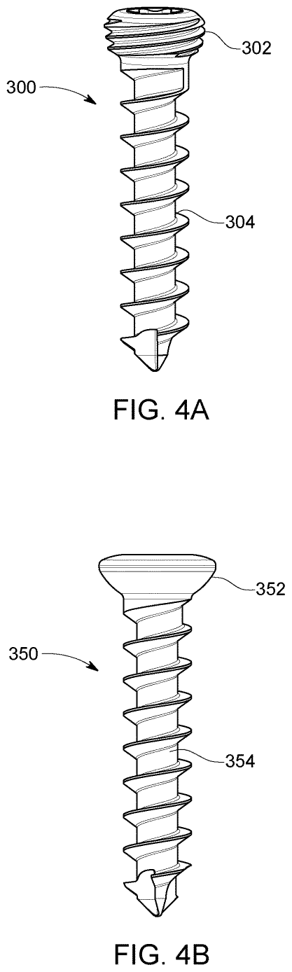

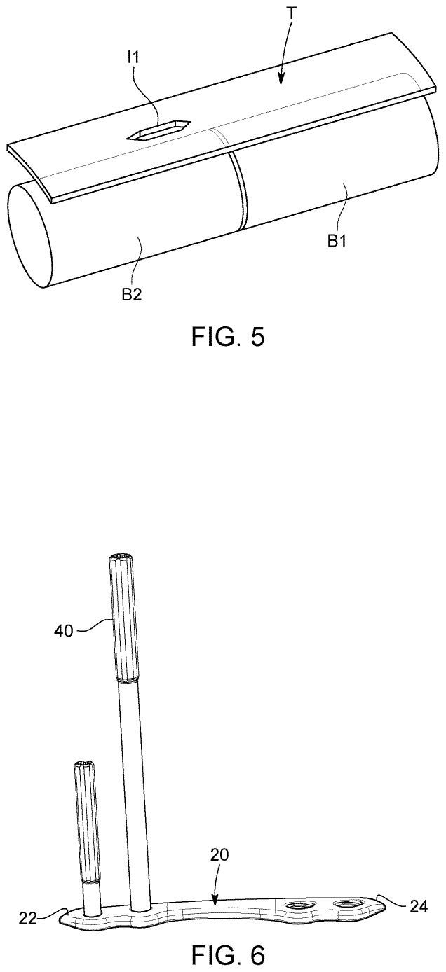

Referring to FIGS. 3 and 4, the system includes multiple sets of anchors 300, 350 to secure the bone plate to the bone segments, as shown in FIGS. 3 and 4. More specifically, FIG. 3 shows a bone anchor 300 having a head 302 and a threaded shaft 304 that extends from the head 302. The head 302 of anchor 300 is threaded to lock into a hole of the bone plate 100. For instance, the threads on the head 302 of the bone anchor 300 are configured to engage the threads on an inner surface of the holes in the bone plate 100. FIG. 4 shows a bone anchor 350 having a head 352 and a threaded shaft 354 that extends from the head 352. Here, the head 352 is not threaded to compress the bone plate 100 toward the bone when the bone anchor fully engages the holes of the bone plate 100. The system may include two bone anchors 300, 350 for the trailing end 22 of the bone plate 20 two bone anchors 300, 350 for the leading end 24 of the bone plate. Each bone anchor used could be a locking bone anchor, a compression anchor, or some mix of locking and compression bone anchors. In one example, the system may include a first outer bone anchor configured to be inserted through the first outer hole at the trailing end 22 and a second outer bone anchor configured to inserted into the outer hole at the leading end 24 of the bone plate, thereby engaging the bone segments adjacent to the trailing end 22 of the bone plate. In addition, the system may include a first inner bone anchor configured to be inserted through the first interior hole at the trailing end 22 and a second inner bone anchor configured to inserted into the interior hole at the leading end 24 of the bone plate, thereby engage the bone segments adjacent the leading end 24 of the bone plate.

FIGS. 5-19 illustrates a method for compressing bone segments using the MIS system described above. Referring to FIG. 5, once the fragment is located via typical visualization techniques, a surgeon or user can form a first incision I1 in tissue T near the bone segments B1, B2. Next, as shown in FIG. 6, in the operating environment, the method includes coupling a first elongated element 40 to the trailing end 22 of a bone plate 20. In this example, the first elongated element 40 may be coupled to the first interior hole of the bone plate 20 using the threaded engagement features described above. In one example, coupling the first elongated element 40 to the trailing end 22 of the bone plate includes threadably coupling the first elongated element 40 to the first interior hole at the trailing end 22 of the bone plate. Optionally, the user may also couple and on outer elongated element to first outer hole of the bone plate 20. This coupling of elongated elements to the bone plate provides a mechanism for the user to grab the assembly begin insertion into the tissue.

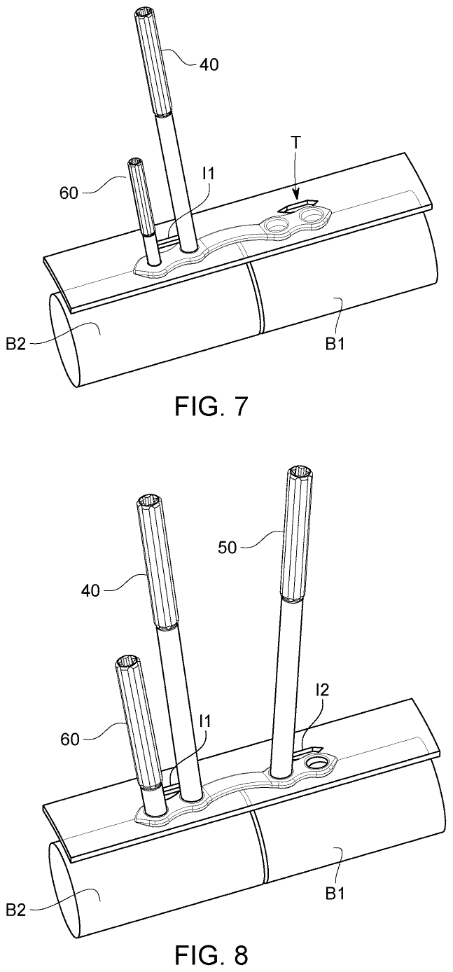

In FIG. 7, the user can insert a leading end 24 of the bone plate through the first incision Il until the leading end 24 of the bone plate is adjacent to a first bone segment B1 and a trailing end 22 is adjacent to a second bone segment B2. The bone segments B1, B2 are divided at the joint or fracture F. The bone plate 20 may be inserted in a distal direction (toward the distal end of the bone, such as a toe or finger, or in a proximal direction (toward the proximal end of the bone). Next, the user can form a second incision I2 in the tissue at or hear bone segments and where the leading end 24 of the bone plate 20 is located.

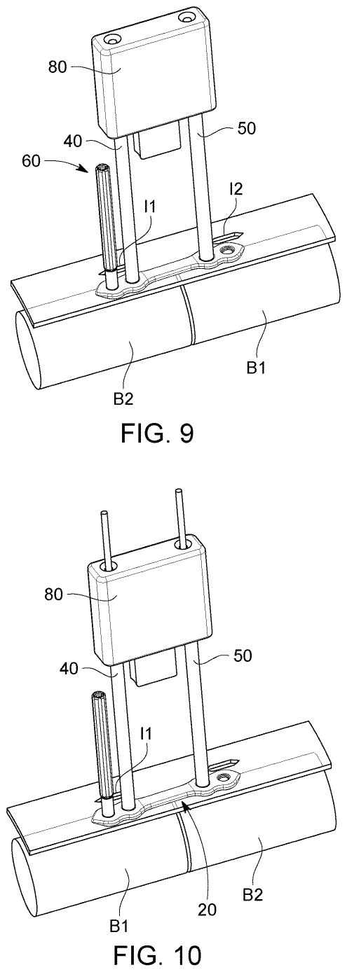

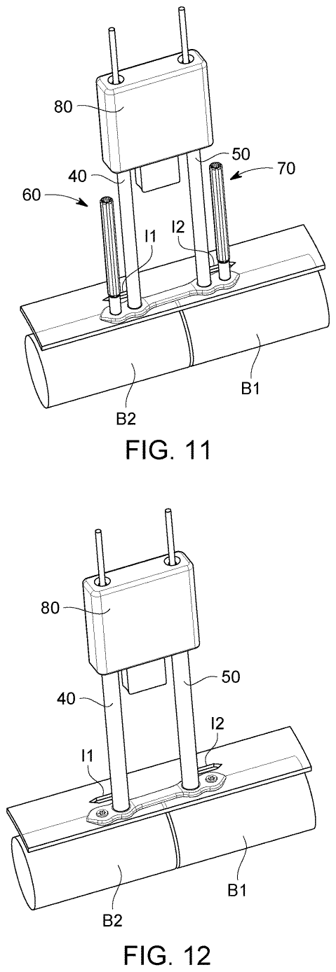

As shown in FIG. 8, the user can couple, through the second incision, a second interior elongated element to the leading end 24 of the bone plate adjacent to the bone segment B2. More specifically, the user can insert the distal end of elongated element 50 through the second incision 12 and threadably couple the elongated element 50 to the second interior hole of the bone plate 24. At this stage, there first and second interior elongated elements are coupled to the bone plate and generally positioned so that their proximal ends are in splayed apart configuration, due to the curvature of the bone plate 20.

In FIG. 9, the device 80 (e.g. a compression jig) is coupled to the proximal ends of the first elongated element 40 and the second elongated element 40 to transition the bone plate 20 from the first curved configuration into a second configuration that differs from the first curved configuration. The user may manually squeeze the two proximal ends together to align the elongated elements 40, 50 with the channels of the jig and slide the jig toward the tissue until the device is fully seated on the elongated elements. In other variations, the jig an be used to engage the proximal ends of the elongated elements 40, 50 and as the jig is slide into position, the elongated elements compress toward each other, flexing the bone plate 20 into a straighter (or nearly straight configuration).

Next, as shown in FIG. 10, first and second stabilization wires are inserted through the jig, elongated elements, and bone plates to engage the bone underneath the bone plate 20, which helps hold the construct in position. In FIG. 11, the second outer elongated element 70 is coupled to the second outer hole of the bone plate 20. Drills, reamers, etc. are then used to form pilot holes in the bone through the outer elongated elements. The outer elongated elements are then removed. The user can then insert a first outer bone anchor, through the first incision I1 into a first outer hole in the trailing end 22 of the bone plate 20 and into the bone segment B2. Then, the user can insert second outer bone anchor, through the second incision 12, into a second outer hole in the leading end 24 of the bone plate and into the second bone segment B1, as shown in FIG. 12 and FIG. 13 (with tissue removed for ease of illustration). This can fix the compressed bone segments together.

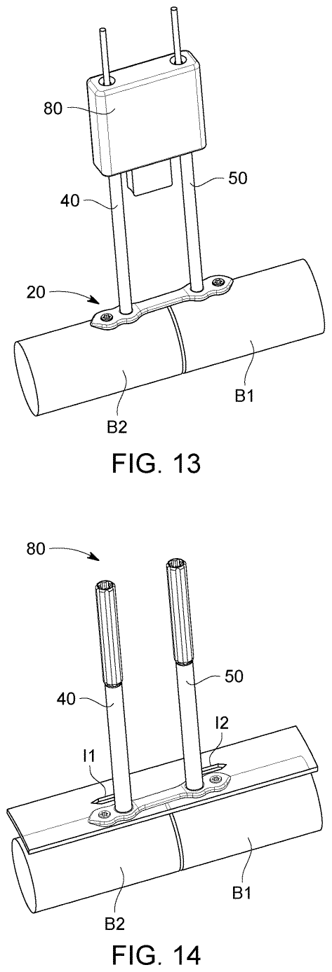

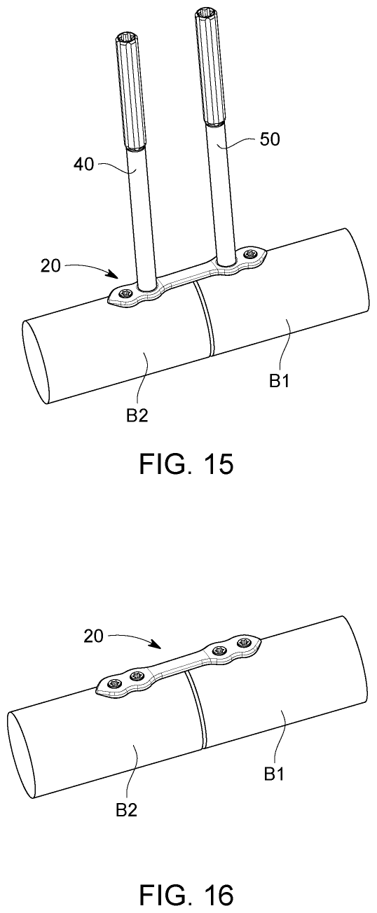

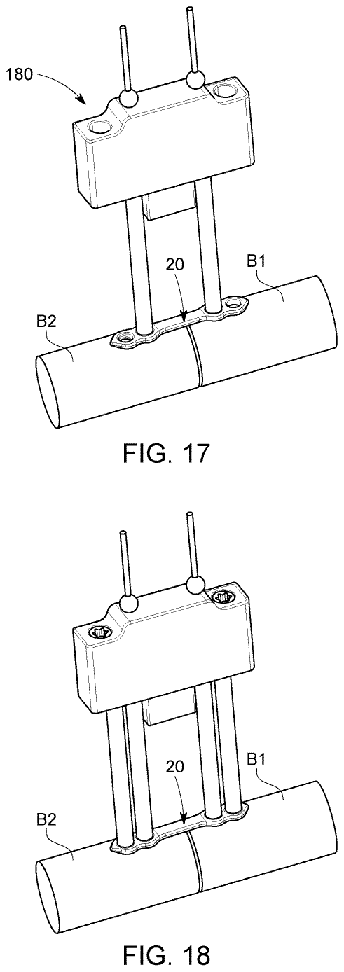

As shown in FIGS. 14 and 15, with two bone anchors in place fixing the bone plate 20 to the bone segments B1, B2 in a generally straighter configuration (e.g. the second configuration), the user can remove the jig 80 from the proximal ends of the first elongated element 40 and the second elongated element 50. This exposes the first and second interior holes of the bone plate 20. The user can then, as shown in FIG. 16, insert a first inner bone anchor (or third anchor) into the first interior hole in the trailing end 22 of the bone plate and into the bone segment B2 and insert second inner bone anchor into the second interior hole in the leading end 24 of the bone plate 20 and into the bone segment B1.

FIGS. 17-19 illustrate an alternative embodiment of the method using a device 180 that includes multiple (e.g., at least four or less than four) engagement elements 1900 (e.g., passages) to receive the elongated elements, in the same manner as described above. In some embodiments, the device 180 can include a center portion 1902 with a height that is greater than a height of each of the lateral portions 1904. The center portion 1902 can include multiple (e.g., at least two) engagement elements 1900a (e.g., at least two inner engagement elements), and each lateral portion 1904 can include at least one engagement element 1900b (e.g., at least one outer engagement element). A portion 1905 can extend downward from a bottom of the device 180 between the elongated element 40 and the elongated element 50. The device 180 can be aligned with and placed over the elongated element 40 and the elongated element 50. The device 180 can be used to compress and straighten the elongated elements. The device 180 can compress the elongated elements toward each other to straighten the elongated elements. The device 180 can also correspondingly flex and straighten the bone plate 20 while the elongated elements are being straightened. In some embodiments, elongated elements can include fixation instruments 1906 for placement into the engagement elements 1900a and/or 1900b. The fixation instruments 1906 can pass through the elongated element 40 and the elongated element 50, and through the bone plate 20 into the bone segment B1 and the bone segment B2. The fixation instruments 1906 can be placed in the inner or outer engagement elements. The engagement elements 1900a can be aligned with inner holes 1907a of the bone plate 20. The engagement elements 1900b can be aligned with outer holes 1907b of the bone plate 20.

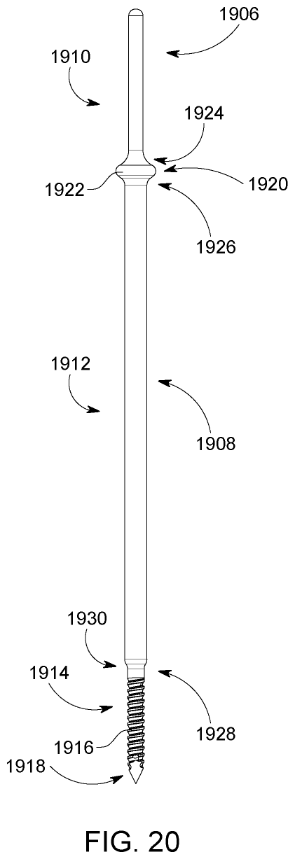

FIG. 20 illustrates the fixation instrument 1906 that can include an elongated tubular body 1908 that includes a first section 1910, a second section 1912, and a third section 1914. The second section 1912 can extend between the first section 1910 and the third section 1914. A diameter of the first section 1910 can be less than a diameter of the third section 1914. The diameter of the second section 1912 can be greater than both the first section 1910 and the third section 1914. The third section 1914 can include threads 1916 and a tapered and threaded tip 1918. The tip 1918 also mimics a 14 millimeter (mm) screw. In some embodiments, the tip 1918 can include a diameter that can range from 2 mm to 3 mm. The first section 1910 can include a diameter that is less than 2 mm (e.g., 1.7 mm).

The second section 1912 can include a first end 1920 that is adjacent to the first section 1910. A protrusion 1922 can be positioned between the first section 1910 and the second section 1912. The protrusion 1922 can be formed from a concave portion 1924 and a convex portion 1926. Opposite to the first end 1920 is a second end 1928 of the second section 1912. The second end 1928 is adjacent to the third section 1914. The second end 1928 can include a convex portion 1930 extending along a circumference of the second end 1928. In some embodiments, the length of the second section 1912 can be greater than the length of the first section 1910. The length of the third section 1914 can be less than the first section 1910.

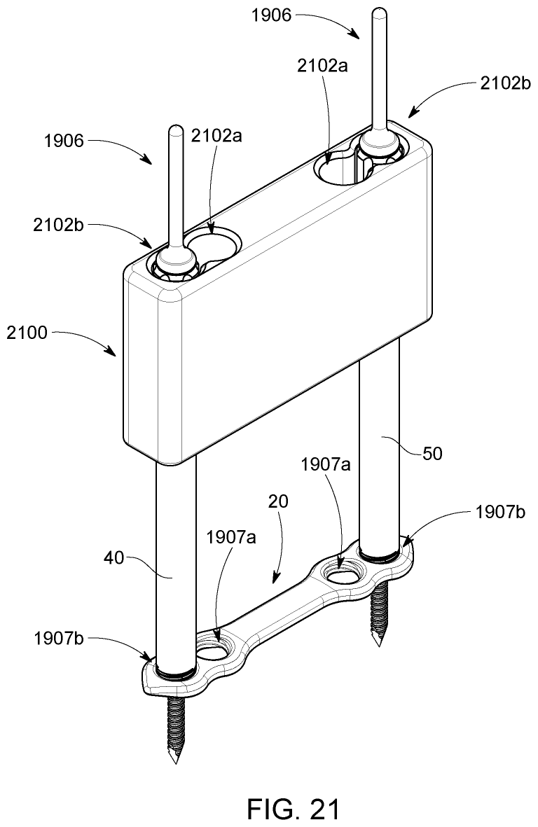

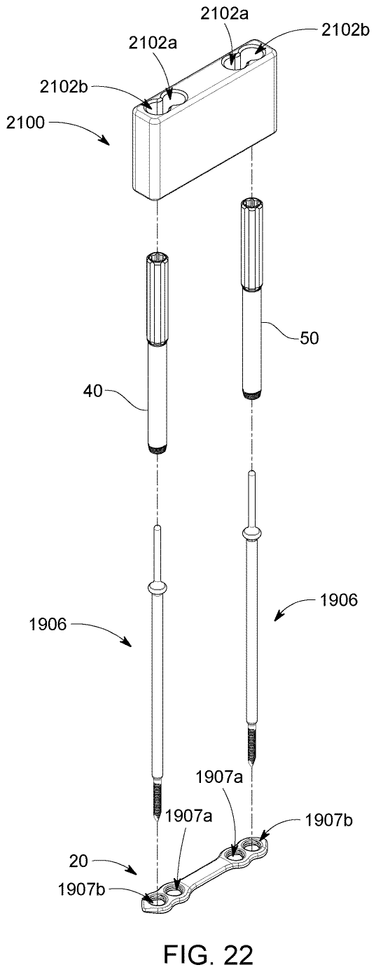

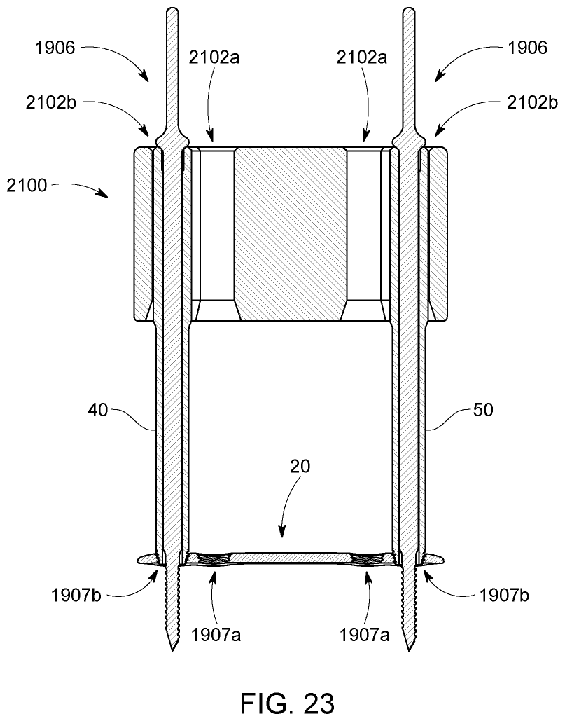

FIGS. 21-23 illustrate a device 2100 that includes four engagement elements, including two inner engagement elements 2102a and two outer engagement elements 2102b. In some embodiments, there may be more than four engagement elements or less than four engagement elements. Each pair of engagement elements (e.g., the engagement element 2102a and the engagement element 2102b) can be in fluid communication with each other, as shown. The device 2100 can compress the elongated elements toward each other to straighten the elongated elements 40, 50. The device 2100 can also correspondingly flex and straighten the bone plate 20 while the elongated elements are being straightened. The fixation instruments 1906 can be placed within the engagement elements 2102a and/or the engagement elements 2102b. The elongated elements can be aligned with the inner engagement elements 2102a and/or the outer engagement elements 2102b. The engagement elements can be aligned with the inner holes 1907a and/or the outer holes 1907b of the bone plate 20.

Wherever possible, the same or like reference numbers are used throughout the drawings and description to refer to the same or like features. It should be noted that the drawings are in a simplified schematic form and are not drawn to precise scale. Certain terminology used in the description is for convenience only and is not limiting. Directional terms such as top, bottom, left, right, above, below and diagonal, are used with respect to the accompanying drawings. Additionally, the term “a”, as used in the specification, means “at least one.” The terminology includes the words above specifically mentioned, derivatives thereof, and words of similar import.

Furthermore, the described features, advantages and characteristics of exemplary embodiments may be combined in any suitable manner in one or more embodiments. One skilled in the art will recognize, in light of the description herein, that the exemplary embodiments can be practiced without one or more of the specific features or advantages of a particular embodiment. In other instances, additional features and advantages may be recognized in certain embodiments that may not be present in all embodiments of the present disclosure.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to a sequential series of reference signs and progression of the blocks in the figures, the method can be implemented in an order as desired.

1. A minimally invasive system for compression of bone segments in an extremity bone, comprising:
a bone plate having a trailing end, a leading end spaced from the trailing end, and a length that extends from the trailing end to the leading end, wherein the bone plate is curved along the length and is configured to flex;
a first elongated element configured to be coupled to the trailing end of the bone plate;
a second elongated element configured to be coupled to the leading end of the bone plate; and
a device configured to be coupled to the first elongated element and the second elongated element while the first and second elongated elements are coupled to the bone plate,
wherein the minimally invasive system has 1) a first configuration where the first elongated element is coupled to the trailing end of the bone plate and the second elongated element is coupled to the leading end of the bone plate so that the first elongated element and the second elongated element are splayed apart with respect to each other, and 2) a second configuration when the device is coupled to proximal ends of the first elongated element and the second elongated element so that the first elongated element and the second elongated element are no longer splayed apart with respect to each other and the bone plate is less curved or no longer curved along the length.

2. The minimally invasive system according to claim 1, wherein the device includes one or more engagement elements configured to be coupled to one or more of the first and second elongated elements.

3. The minimally invasive system according to claim 2, wherein the device is a jig having a first engagement element configured to receive the first elongated element and a second engagement element separate configured to receive the second elongated element.

4. The minimally invasive system according to claim 3, wherein the first engagement element is a first channel, and the second engagement element is a second channel.

5. The minimally invasive system according to claim 4, wherein the jig defines the first channel and the second channel.

6. The minimally invasive system according to claim 3, wherein the device has a third engagement element and a fourth engagement element.

7. The minimally invasive system according to claim 6, wherein the third engagement element is a third channel, and the fourth engagement element is a fourth channel.

8. The minimally invasive system according to claim 1, wherein the elongated elements are drill guides.

9. The minimally invasive system according to claim 1, wherein the device is a tool attachable to the first elongated element and the second elongated element, wherein actuation of the tool causes the system to transition from the first configuration into the second configuration.

10. The minimally invasive system according to claim 9, wherein tool includes first and second threaded portions, rotation of either cause the tool to transition the system from the first configuration into the second configuration.

11. The minimally invasive system according to claim 9, wherein tool includes first and second cam portions, rotation of either cause the tool to transition the system from the first configuration into the second configuration.

12. The minimally invasive system according to claim 1, wherein the device includes a first bore configured to receive a stabilization wire, and a second bore configured to receive another stabilizing wire.

13. The minimally invasive system according to claim 1, wherein the bone plate has a central bridge that extends between the trailing end and the leading end.

14. The minimally invasive system according to claim 1, wherein the bone plate has a hole at the trailing end of the bone plate and a hole at the leading end,
wherein the first elongated element has a proximal end, and a distal end configured to be coupled to the bone plate at the hole at the trailing end,
wherein the second elongated element has a proximal end, and a distal end configured to be coupled to the bone plate at the hole at the leading end.

15. (canceled)

16. The minimally invasive system according to claim 1, wherein the trailing end of the bone plate includes a first interior hole and a first outer hole, and the leading end includes a second interior hole and a second outer hole.

17. (canceled)

18. (canceled)

19. A method, comprising:
positioning a bone plate through a first incision with a first elongated element coupled to the bone plate so that the bone plate spans bone segments;
forming a second incision in a location spaced from the first incision;
coupling a second elongated element to the bone plate through the second incision;
coupling a device to the first elongated element and the second elongated element to flex the bone plate from a first configuration into a second configuration;
inserting bone anchors through the first and second incisions to couple the bone plate to fractured bone segments; and
removing the device from the first elongated element and the second elongated element to transition from the second configuration toward the first configuration to compress the fractured bone segments together.

20. The method according to claim 19, further comprising, before positioning a bone plate through a first incision, forming the first incision in tissue near a foot bone.

21. The method according to claim 19, wherein inserting bone anchors through the first and second incisions to couple the bone plate to fractured bone segments includes inserting one or more anchors through the first incision to the couple a trailing end of the bone plate to one of the bone segments and inserting one or more bone anchors through the second incision to the couple a leading end of the bone plate to the other of the bone segments.

22-24. (canceled)

25. The method according to claim 19, wherein the first configuration is when the bone plate is curved and the second configuration is when the bone plate is not curved, wherein removing the device from the first elongated element and the second elongated element causes the bone plate to curve to transition compress the bone segments together.

26. A method, comprising:
forming a first incision in tissue near fractured bone segments;
coupling a first elongated element to a trailing end of a bone plate, the bone plate having a first curved configuration;
inserting a leading end of the bone plate through the first incision until the leading end of the bone plate is adjacent a first bone segment and a trailing end is adjacent to a second bone segment;
forming a second incision in tissue near where the leading end is located;
coupling, through the second incision, a second elongated element to the leading end of the bone plate adjacent to the second bone segment;
coupling a jig to proximal ends of the first elongated element and the second elongated element to transition the bone plate from the first curved configuration into a second configuration that differs from the first curved configuration;
inserting a first anchor, through the first incision into a first outer hole in the trailing end of the bone plate and into the first bone segment;
inserting a second anchor, through the second incision, into a second outer hole in the leading end of the bone plate and into the second bone segment; and
removing the jig from the proximal ends of the first elongated element and the second elongated element to transition from the second configuration toward the first curved configuration, thereby compressing the first bone segment and the second bone segment together.

27. The method according to claim 26, wherein the bone plate has a first interior hole at the trailing end and second interior hole at the leading end of the bone plate, wherein coupling the first elongated element to the trailing end of the bone plate includes threadably coupling the first elongated element to the first interior hole at the trailing end of the bone plate.

28. The method according to claim 27, wherein coupling the second elongated element through the second incision to the leading end of the bone plate includes threadably coupling the second elongated element to the second interior hole at the leading end of the bone plate.

29. The method according to claim 26, wherein coupling the jig to the proximal ends of the first elongated element and the second elongated element further includes positioning the jig so that a first channel receives a proximal end of the first elongated element and a second channel of the jig receives a proximal end of the second elongated element.

30. The method according to claim 26, after inserting the first anchor and second anchors, further comprising removing the first elongated element and the second elongated element from the bone plate.

31. The method according to claim 26, further comprising coupling a first outer elongated element to first outer hole of the trailing end of the bone plate.

32-34. (canceled)

35. The method according to claim 26, after coupling the jig to the proximal ends of the first and second elongated elements, inserting first and second stabilization wires through the jig.

36. A method, comprising:
forming a first incision and a second incision in tissue near a joint or fracture located between a first bone segment and a second bone segment;
inserting a bone plate through the first incision until the bone plate is adjacent the first bone segment and the second bone segment, wherein the bone plate is in an elastically deformed state;
inserting a first anchor through the first incision into a first hole the bone plate and into the first bone segment;
inserting a second anchor through the second incision into a second hole the bone plate and into the second bone segment; and
allowing the bone plate to transition from the elastically deformed state into a non-deformed original state, thereby compressing the first and second bone segments.

37. The method according to claim 36, further comprising causing the bone plate to transition from a first configuration, where the bone plate is not elastically deformed, into a second configuration, where the bone plate is in the elastically deformed state.

38. (canceled)

39. The method according to claim 36, wherein allowing the bone plate to transition occurs in situ when the bone plate is coupled to the first and second bone segments.

40. (canceled)

41. The method according to claim 36, wherein inserting the bone plate through the first incision includes inserting a leading end of the bone plate through the first incision until the leading end of the bone plate is adjacent a first bone segment and a trailing end is adjacent to a second bone segment, wherein the trailing end is coupled to a first elongated element.

42.-45. (canceled)
